(19) United States
(12) Patent Application Publication
(10) Pub. No.: US 2009/0201172 A1
(43) Pub. Date: Aug. 13, 2009

(54) Miniature Physiological Telemeter

(75) Inventor: David J. Edell, Lexington, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: INNERSEA TECHNOLOGY, INC., Bedford, MA (US)

(21) Appl. No.: 11/920,580

(22) PCT Filed: May 16, 2006
(86) PCT No.: PCT/US2006/019018

§ 371 Date: Mar. 31, 2009

Related U.S. Application Data
(60) Provisional application No. 60/681,520, filed on May 16, 2005.
(60) Provisional application No. 60/681,887, filed on May 17, 2005.

(51) Int. Cl. G08C 19/00; A61B 5/00

(52) U.S. Cl. 340/870.3; 600/300

(57) ABSTRACT

A low power miniaturized telemeter (25, 40-42) provides data from a monitored subject at internal or external locations. A charge integration and pulse stream encoding (30) in the telemeter (25, 40-42) contributes to reduced power consumption. A transmitter (29) in the telemeter (25, 40-42) may be omnidirectional to permit operation without physical obstruction or limitations to movement. A receiver (22) collects transmitted information and may have an adaptive threshold pulse detector to permit further reductions in power usage. The telemeter (25, 40-42) can multiplex monitored parameters on a time division basis to permit trans-mission of multiple data channels. Individual telemeters may have unique transmission frequencies to permit multiple telemeters to be used concurrently without interference. A self-contained power source in the telemeter (25, 40-42) permits long term operation at low power without the need of replacement. The telemeter (25, 40-42) can be packaged to accommodate a number of applications, such as by permitting adjustable density.

### CROSS REFERENCE TO RELATED APPLICATIONS

This application claims the priority of U.S. Provisional Application Nos. 60/681,520 filed May 16, 2005 and 60/681,887 filed on May 17, 2005 both of which were entitled MINIATURE PHYSIOLOGICAL TELEMETER, the whole of which is hereby incorporated by reference herein.

### STATEMENT REGARDING FEDERALLY SPONSORED RESEARCH OR DEVELOPMENT

N/A

### BACKGROUND OF THE INVENTION

While advanced instrumentation is readily available for physiological monitoring and research, this instrumentation is generally not portable and it must be connected to a patient or subject with wire leads. For many studies with sedentary subjects, direct lead recordings are acceptable. However, when it is desirable to have patients or subjects exercise while being monitored, wired attachments are problematic, because wiring can dislodge sensors or cause artifacts in recordings. If a large number of monitoring points are of interest during physical activities, particularly during sports, wired systems are generally not effective.

Prior art biotelemeters are very simple systems with poorly controlled sensors, gains, linearities, center frequencies and harmonics. These systems also exhibit relatively high power consumptions. Some prior art systems are as tall as six stacked nickels. At high acceleration levels, not only are forces much higher for these prior art, single channel systems, but torque is extreme for a unit of that height. Prior art biotelemeters usually rely on variable inductances, resistances or capacitances that are cleverly embedded in a transmitter, but this limits the types of sensors that can be telemetered.

In sports medicine and clinical rehabilitation, where it may be useful to have athletes engage in their normal activities, such as pole vaulting, hurdling or pitching a baseball, wire-connected systems cannot be used, even with belt worn telemetry. Furthermore, if such monitoring is to take place over extended times or with sensors attached to points of high acceleration, wired systems are impossible to use effectively.

Wireless telemeters are known for recording data from mobile subjects. For example, multichannel telemetry systems for providing bioelectric signals are commercially available. However, the instrumentation packages are relatively large, having a typical size of that of a hand-held PDA or larger, such as approximately 5×2×1 inches, for example. These instrumentation packages can have a weight on the order of 1 pound or more. Typically, they are worn on a belt and wired to a distributed electrode location. These types of devices are typically limited to applications involving slow movements or low accelerations, where power usage is somewhat less of a concern.

Other wireless telemeter modules are known for recording bioelectric signals. For example, a 12-channel wireless module for recording single channel bioelectric signals is commercially available. A single unit weighs approximately 30 grams and has a dimension of approximately 54×46×15 mm. Battery lifetime for the wireless module is approximately 15 minutes, where the module has a communication range of approximately 50 M.

One of the biggest challenges in providing a wireless telemetry module for monitoring physiological parameters is power usage. Typically, a wireless system such as may include a radio frequency (RF) transmitter consumes a significant amount of power, such that implantable or long-term monitoring of physiological parameters is impractical. This is especially true if there is a relatively large separation between the transmitter and receiver, which is often desirable in applications for monitoring physiological data for subjects in motion, especially with large physical accelerations.

What is needed is a new wireless physiological instrumentation system that can be used on subjects engaged in physical activities, in clinical rehabilitation, occupational therapy, sports medicine studies and specific training for prevention of injuries.

### BRIEF SUMMARY OF THE INVENTION

The invention is directed to a wireless, unobtrusive, multi-point, multi-sensor physiological measurement instrumentation system that can be used on subjects engaged in physical activities in clinical rehabilitation, occupational therapy, sports medicine studies and specific training for prevention of injuries. These systems also find applications for patients recovering from certain surgeries, such as limb replantation. Such a system provides methods and apparatus for acquiring high quality, multi-variable physiological data from many locations on an active subject. The data can be archived in any Ethernet-connected computer for analysis using existing commercial or laboratory software.

The disclosed instrumentation system provides a readily useable, reasonable cost alternative to wired systems for use with active subjects. Embodiments of the invention include, e.g., small, self-contained instrumentation “buttons” that are capable of transducing multiple physiological parameters and transmitting the data continuously to a nearby receiver and computer. These devices are sufficiently small and lightweight to remain in place while transmitting reliable data, even during movements involving high accelerations.

The system can be thought of as having two parts: a physiological telemeter and a receiver. The physiological telemeter takes in, preferably, a plurality (e.g., four) sensor signals (such as voltages), encodes the signals in pulse representations (such as signals in which times between pulses are proportional to the signal amplitudes), and transmits the pulses as a wireless signal, e.g., by radio frequency (RF) transmissions, to a receiver. The sensor voltages can be from virtually any type of sensor now available or that will be available in the future.

The purpose of an individual physiological telemeter according to the invention is to provide continuous, simultaneous telemetry of multiple physiological variables from one location on the skin or inside of an animal or human. Multiple physiological telemeters can be operated simultaneously to acquire multiple physiological variables from multiple locations.

The overall size and weight of the physiological telemeter is dictated primarily by the battery. It is the size and weight of the Physiological telemeter that must be minimized in order to be able to use the technology in a wide variety of medical applications. This need for minimization of size and weight is because: 1) the size and weight determine the shear forces that would be generated by acceleration of a body part when used on an active subject; 2) size determines the minimum area of skin, or the minimum size body cavity, with which the devices can be used; and 3) size determines the cosmesis, or user acceptability factor.

Shear forces are important because they define the level of adhesion that is required for fixing the physiological telemeters to the skin. Also, internally, the forces that will be generated by accelerations against the connective tissue and sutures used for stabilization will be proportional to the weight of the device. Perhaps of greater fundamental importance, these shear forces will move a cutaneous mounted device relative to the underlying tissues or an implanted device relative to the structures of interest. Obvious examples where this would be critical would be when recording EKG or EMG, or heart, chest or gut sounds. In all of these cases, motion of the device due to accelerations would greatly distort the signals of interest. By minimizing size and weight, thus decreasing the mass, these effects are minimized, thereby enabling the measurements.

For long term monitoring applications, minimizing weight and size promotes cosmesis but more importantly also minimizes the aggressiveness of the adhesives that need to be used for cutaneous mounting of the devices. Aggressive adhesives can lead to skin irritation or even ulceration. In addition, when used under extreme conditions of exercise and hot, humid conditions such as might be found during football workouts for example, the adhesives are challenged by sweat and oils, so small, lightweight devices would be advantageous.

In addition to aggressive engineering design efforts to reduce circuit power requirements, the overall circuit concept was developed to dissipate power approaching the minimum theoretical power possible, with clear tradeoffs of power, bandwidth, and transmission distance resulting. The circuit approach is as follows.

While synchronous or asynchronous encoding may be used, an asynchronous pulse position modulation scheme has several advantages. Being based on integration of a current representation of the signal of interest, asynchronous encoding minimizes the pre-amplification requirements for the signal, and inherently reduces noise because noise that is higher than the encoding bandwidth will be averaged out by the integration process. Both of these features allow reduction in circuit power requirements. Also, the asynchronous encoding method minimizes required transmission power because only one pulse is used to transmit one signal element, and because no clock circuitry is required as it is “self-clocked” by the cycling of the integrator.

Second, signal conditioning is minimized as mentioned above by converting signals to current representation with minimal conditioning. Since the integration process performs a low pass filter function, “anti-aliasing” filters are not as essential as they are for a more traditional sampled voltage conversion. A typical signal path, for amplifying a bioelectric signal for example, consists of a single stage amplifier with simple band limiting passive elements. Voltage to current conversion is then accomplished by simple resistive conversion. The resistance for a particular variable can be easily adjusted to ensure proper dynamic range of the resulting signal current. Once the signal current is generated, it is simply passed through a multiplexing switch to the integrator/encoder.

The encoding process simply consists of a low noise current integrator that resets itself once the output crosses a threshold voltage that is set on a comparator. The comparator resets the charge on the integration capacitor. During this reset time interval, the transmitter is turned on briefly.

Use of pulse encoding can also allow a second level of multiplexing. While the first level of multiplexing discussed above combines multiple signals sensed from multiple sensors connected to or as part of a single physiological telemeter, multiple physiological telemeters can be operated simultaneously by choosing different frequencies for each physiological telemeter. For example, multiple RF physiological telemeters, each operating on a distinctly different RF frequency, can transmit simultaneously to a receiver. The receiver can have multiple demodulators that are each sensitive to a specific frequency of RF. In similar fashion, if sound is used instead of RF, different frequencies of sound can be used. Also, if light is used, different frequencies of light will allow this second level of multiplexing. However, applications in which optical communications are available may be limited to the extent that line of sight communications are unavailable. Further, combinations of light, sound and RF can all be used simultaneously if necessary to allow more options. By operating on different frequencies, the physiological telemeters that are being used simultaneously do not need to synchronize with each other. Again, this saves circuit complexity which saves power which allows minimization of size and weight—the primary design objective.

With a small size, the physiological telemeter achieves a useful reduction in the separation distance of probes used to collect physiological data. By placement of probes in a more proximate location to each other, noise from differential sources, or noise that impacts separate probes, is reduced or eliminated. The common mode rejection ratio (CMRR) of the device is improved, leading to further reductions in size permitted by omitting additional noise reduction circuitry. Prior, larger dimensioned devices are unable to take advantage of this feature due to their greater size.

Packaging of the physiological telemeters is critical to maintaining the low mass, small size. Traditional approaches would be to put the system in a urethane or silicone, for example. The packaging should be flexible to allow conformation of the device to the skin or to an organ. Packaging must also stabilize electrodes to ensure proper contact with the target. In order to protect sensitive or fragile circuit elements, it is preferred to use a stiff encapsulant. However, stiff encapsulants can be heavy and will interfere with the conformation of the device to various shapes such as a muscle surface or the chest over the heart. Packaging must also be protective against moisture and salts. Good choices for flexibility and electrical protection are urethanes and silicones, for example, as they withstand saline environments better than most encapsulants. Epoxies are another possibility but are generally stiff. All are relatively heavy and are denser than tissue, which would make the assembly heavier than desirable. One method to counter this would be to include foamed encapsulants (by rapid stirring, blowing in CO₂, or by including a chemical foaming agent, for example) or glass microspheres in the formulation of the encapsulant. Glass microspheres have the advantage of adding structural stability from the glass spherical shape while reducing the density considerably since they are mostly air. So, over the circuit, a microsphere filled version of encapsulant can be used to enhance the structure of a flexible silicone, for example, while in the electrode region where flexibility is needed the microspheres can be reduced or eliminated to allow full flexibility of the base material. In the case of a flat, circular design, the circuits are on a PC board or flexible PC board and would be encapsulated with the glass microsphere material, while outside the diameter of the PC board, where the outer pickup electrode is typically located, non-glass microsphere filled material would be used to ensure conformation to the target structure.

The interface between the physiological telemeter and the skin is critical to its function, particularly for long term use in monitoring patients for autonomic dysreflexia or for monitoring performing athletes. One approach would be to use a flexible, stretchable adhesive tape. However, if the subject is excessively hairy (some people, most animals), tape usually requires shaving which may be unacceptable. If an adhesive is used, it may be worked through the hair to bind the hair together and thus provide a stable area of fixation. If the adhesive is made conductive (for example, a collagen adhesive gel with added salt, but there are many other possibilities), then electrodes needed for recording biopotentials can record through the adhesive. If the conductivity of the adhesive is too high, the electric potentials on the skin surface will be shorted out thereby reducing or eliminating the potentials of interest. If the conductivity is too low, then the electrode-electrolyte impedance and drift will be too high. If the conductivity is roughly what the conductivity of the skin is, then the potential distribution will be essentially unchanged for reasonable thicknesses of adhesive. This is probably ideal and can be achieved using a variety of water based adhesive gels, for example.

Applications of the small, lightweight physiological telemeters are many. For example, implantable physiological telemeters can be operated by coupling RF power to either directly operate the implantable circuits or to recharge batteries that operate the circuits may be realized to extend useful device lifetimes. Implanted physiological telemeters can sense and transmit electrical, pressure, sound, thermal, optical or chemical signals depending on the selected transducers. These can be used for a wide variety of physiology studies, monitoring, and diagnostics. They could also be used in closed loop control of manipulations of the physiological system. For example, heart rate, blood pressure, hydration, osmolarity, etc., could be monitored and transmitted out to control infusion of drugs that help control the cardiovascular system, or to control autonomic dysreflexia. There are many other possibilities. Surface mounted physiological telemeters can be used to telemeter data from many muscles (through EMG sensing), temperature, and sound, for example, to monitor exercise stress and fatigue in performing athletes. Inclusion of other physical measures such as joint angles by using magnetic, stretch, optical sensors, resistive encoders, etc., would permit a complete motion analysis system to be assembled, which, in addition to monitoring motion, could monitor the muscles that actuate the joints at the same time. By monitoring many muscles and joints simultaneously in a performing athlete of any kind, e.g., human or animal, e.g., horse, it will be possible to better understand sports biomechanics, and will eventually lead to a better understanding of how to properly condition athletes, rehabilitate injured athletes, and train athletes to avoid injury. In addition, during performances, critical muscles understood to be the first to fatigue for a particular athlete, or in general, could be monitored during the performance so the athlete could be informed of impending fatigue which could reduce the level of performance or lead to serious injury. For example, the shoulder and leg muscles of a baseball pitcher could be monitored for fatigue and over-use indicated by changes in EMG and sound signal frequencies, and by temperature. Clinically, monitoring surgical patients for proper recovery of perfusion following vascular repair for example could be accomplished by monitoring temperature and oxygenation (using pulse oximetry sensing, for example).

Thus, in general, the invention is directed to an apparatus for measuring a body parameter and providing data communications related to the parameter. The apparatus may include a transducer coupled to the body for sensing the body parameter. A converter coupled to the transducer takes the transducer signal and produces a pulse stream that can be transmitted with a transmitter using low power techniques. The transmitter may be omnidirectional to communicate data related to the body parameter without mechanical interference or limitations on movements of the body. A receiver communicating with the transmitter receives and decodes the communicated data, and can operate on one or more channels, with one or more telemeters. Since the receiver is located some distance from the transmitter, there is no mechanical interference with the subject and no movement limitations are placed on the body. For an inanimate subject, the flexibility of receiver location, in placement and distance, permits the use of the telemeter system in locations that are impractical to reach for any reason.

According to one aspect of the present invention, there is provided a method for measuring a body parameter and providing data communications related to the parameter. The method may include sensing the body parameter and generating a measurement signal, which is converted to a pulse stream. The pulse stream is transmitted to a receiver, which decodes the received signal and pulse stream to obtain the desired data. The receiver may operate on one or more channels to permit multiple telemeters, and may also decode communications that are time division multiplexed from one or more telemeters.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIG. 1A shows a signal path for a single physiological telemeter according to the invention from a skin interface to a data archive computer;

FIG. 1B shows a circuit block diagram for a charge integration circuit;

FIG. 2 is a block diagram of three physiological telemeters transmitting on unique frequencies to an Ethernet interface;

FIGS. 3A-3C show summaries of three embodiments of physiological telemeters according to the present invention;

FIG. 4 shows elements of a miniature physiological telemetry system according to the invention in a hypothetical sports medicine study;

FIG. 5 shows an overview of a signal path for an asynchronous pulse train representation of multiple signal channels according to the invention;

FIG. 6 is a cross-sectional illustration of a surface-mounted telemeter according to the present invention;

FIG. 7 is a cross-sectional block diagram view of transducers for use with the present invention;

FIGS. 8A-8C illustrate a packaging embodiment for a telemeter in accordance with the present invention;

FIGS. 9A-9B illustrate another packaging embodiment for a telemeter in accordance with the present invention; and

FIGS. 10A-10B show a prototype physiological telemeter and a receiver composed of commercial modules and existing lab-built circuits.

### DETAILED DESCRIPTION OF THE PREFERRED EMBODIMENT OF THE INVENTION

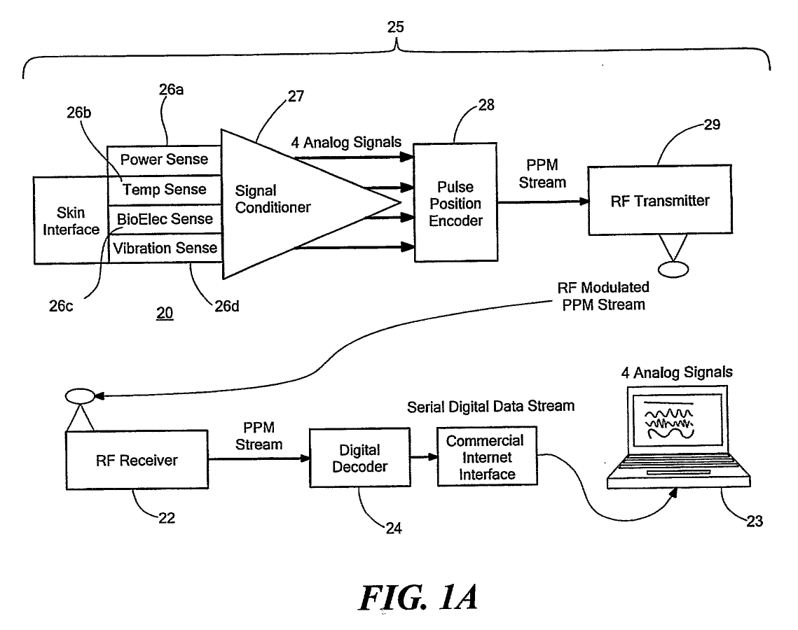

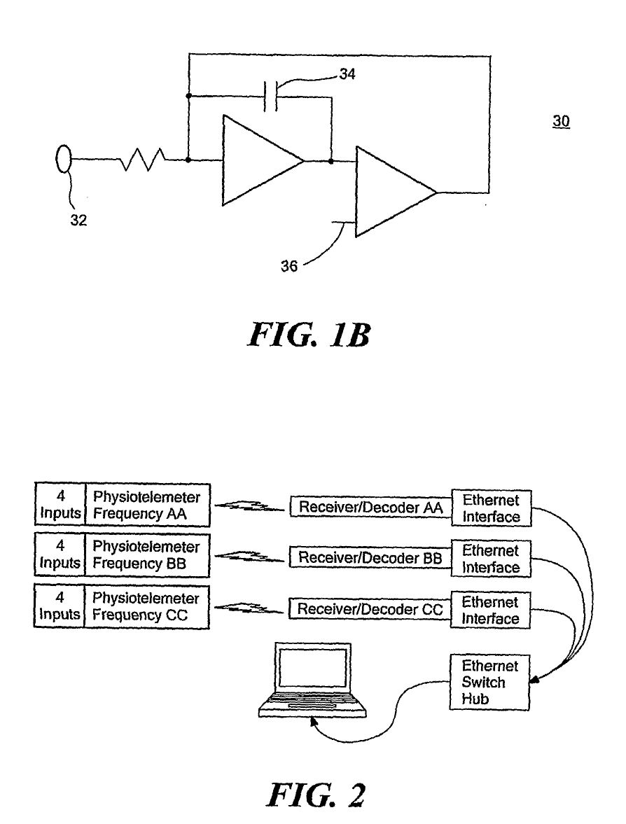

A miniaturized, self-contained, physiological telemetry device is located in or on a body in an area of interest. This device, called a physiological telemeter, transmits on a unique RF frequency allowing multiple physiological telemeters to be used simultaneously. Each physiological telemeter operates autonomously from a small, rechargable coin-cell battery, which accounts for roughly half the mass of the physiological telemeter. A block diagram of a physiological telemeter system 20 of the invention is shown in FIG. 1A. A telemeter 25 includes all of the components for sensing physiological parameters and transmitting data to a receiver 22. Telemeter 25 may include a number of transducers, such as transducers 26A-26D. The physiological parameters obtained through transducers 26A-26D are applied to a signal conditioner circuit 27, which can be a conversion circuit specifically tailored for low-power, low-noise amplification and conditioning of physiological parameter signals. For example, signal conditioner 27 may convert signal outputs from transducers 26A-26D to current signals that can have a high degree of noise immunity while consuming little power. The conditioned signals are applied to an encoder 28 that produces a pulse stream representation of the conditioned signals. One technique for producing a pulse stream representation of current signals is to use a charge integration circuit, described in greater detail below. The pulse stream output from encoder 28 has very sort duration pulses to conserve power while encoding signal information based on pulse interval. Encoder 28 can multiplex a number of signals, such as the four illustrated, so that a single pulse train provides pulse position modulation encoding the information contained in the four input signals. That is, the pulses representing each signal are interleaved by tie division basis to provide a single pulse stream that encodes all four channels. The pulse stream is applied to a transmitter 29 that transmits the pulse encoded information to a receiver 22. Transmitter 29 may use power optimization transmission techniques to further reduce power usage for telemeter 25. For example, transmitter 29 may take advantage of the applied pulse stream to tailor the transmission modulation for low-power operation.

Transmitter 29 and receiver 22 are separated by a relatively large distance that permits relative motion between transmitter 29 and receiver 22 without loss of signal fidelity. In addition, the subject to which telemeter 25 is coupled has a free range of movement without interference from wires or a closely spaced receiver. Accordingly, system 20 permits monitoring of physiological parameters, while a subject is actively engaged in a given set of motions or activities without any substantial interference from the monitoring equipment. Data is also collected in system 20 for analysis with techniques such as may be available with computational or biometric programs that may be provided on a computer 23. System 20 accordingly may be used for immediate feedback related to given activities monitored with telemeter 25.

A significant reduction in power can be realized for a telemeter in accordance with the present invention by reducing collected and transmitted signal power. FIG. 1B illustrates a charge integration circuit 30 that receives a signal 32 that encodes transducer collected data. The signal is in the form of a current signal, which provides a number of advantages for improved signal fidelity and resistance to noise, for example. The current signal is applied to a charge integration capacitor 34, which is charged over an interval that is specific to the data encoded by the current signal. Once the charge on capacitor 34 reaches a threshold 36, capacitor 34 is discharged, or the integration function is reset, to permit another encoding cycle to commence. The time interval between resets encodes the current signal information, and a pulse train with rising edge intervals is generated that represents the intervals. The pulse train is fed to a multiplexer/transmitter that sends the pulse train to a receiver. The nature of the pulse encoding, also referred to as pulse position encoding, produces a low power representation of the data to be communicated, so that the transmitter consumes a minimal amount of power.

In another embodiment, multiple telemeters transmit on separate frequencies to separate receivers, as shown in FIG. 2. In each embodiment, a number of signals are multiplexed together, for example as pulse positions in a pulse stream, to be transmitted to a receiver. Accordingly, the disclosed system and method are capable of operation with multiple multiplexed channels to deliver a large amount of data with very little power consumption. The data paths converge in an interface, which can be a network interface such as an Ethernet interface. The network interface permits simple data collection of multiple data channels. In one embodiment, the data are collected by a multi-port Ethernet switch. Total harmonic distortion was <0.5%, probably due to a slight aliasing effect caused by a marginal sampling rate evidenced by the rolling waveform.

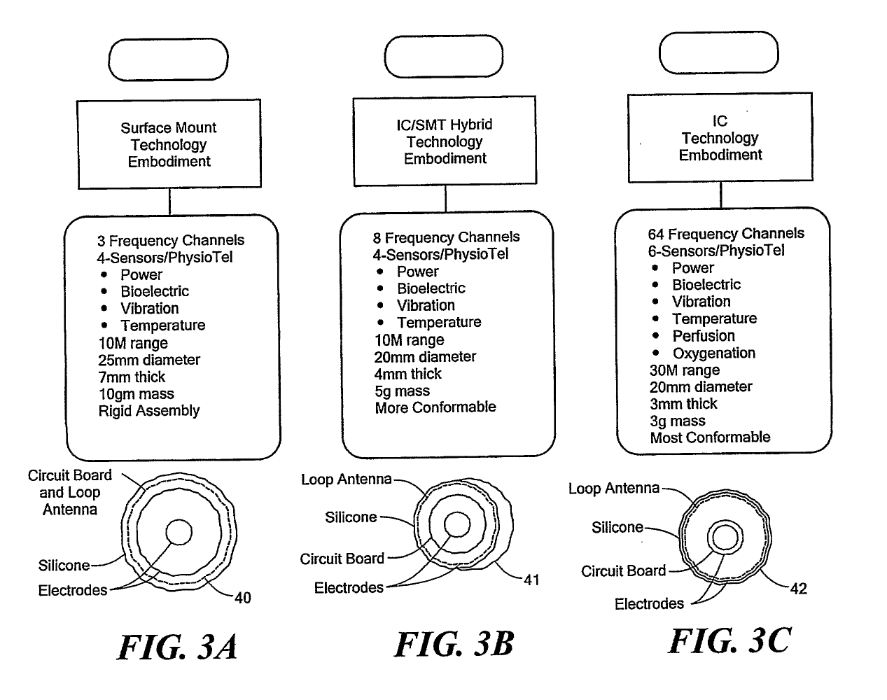

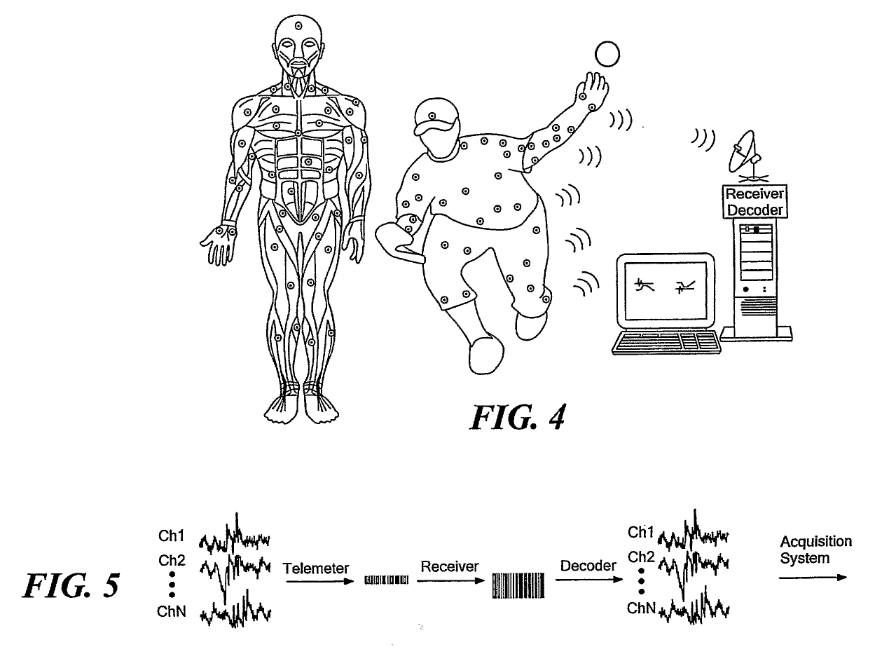

Referring to FIGS. 3A-3C, various embodiments of a telemeter according to the present invention are illustrated. The telemeters transmit on unique frequencies whereby the telemetered data elements are received, decoded and then multiplexed, e.g., into an Ethernet format, with or without the use of a relay or repeater, via a commercial interface. One embodiment of a physiological telemeter is about the diameter of a US nickel (˜22 mm), about twice as thick (3-4 mm), but with lower mass (˜3 g). One key to accomplishing the miniaturization is to minimize power, which minimizes the battery size. In another embodiment, some or all of the components of the physiological telemeter are implemented on an integrated circuit, which can operate on even lower power. In one embodiment, up to 64 individual physiological telemeters can be used simultaneously, each transmitting on a separate frequency.

A diagram showing various elements of one embodiment used for a high-speed activity analysis is shown in FIG. 4. In the pitching activity shown, small, low mass, telemeters are attached to the skin surface over physiologically distinct areas and monitored during an activity to provide time-synchronous, multi-point, multi-variable data for analysis of the activity for rehabilitation or training. In this embodiment, the telemeters would be subjected to, high accelerations during complex pitching movements.

Referring again to FIG. 2, a 3-channel system is illustrated, in which each physiological telemeter encodes and transmits data from three biosensors and a low battery sensor. The three biosensors can be, for example, bioelectric (optimized for surface electromyogram or EMG), acoustic (optimized for muscle EMG), and skin temperature. Other biometric inputs can include sounds from heart, lung or muscle, skin perfusion, oxygenation, pulse, joint angle or other mechanical transducers, ambient temperature, pressure or any variable that can be transduced to a voltage, current, resistance, capacitance or other analog. Table 1 summarizes several example physiological variables according to a metric that can be sensed to monitor the physiological variable.

TABLE 1
Example physiological variables organized according to the form of energy sensed.

| Transducer type | Electrical | Acoustical | Thermal | Optical | Mechanical |
|---|---|---|---|---|---|
| Physiological | Brain (EEG) | Muscle | Temperature | Oxygenation | Joint Angle |
| data: Example 1 | | | | | |
| Example 2 | Heart (EKG) | Lung | Perfusion | Arterial Pulse | Pressure |
| Example 3 | Muscle (EMG) | Heart | | | |

Thus, each telemeter can be a multi-sensor array transmitting, e.g., four physiological variables. The availability of this multi-variable data should enhance data interpretation, because the variables of primary interest can be kept within the context of the physiological environment from which they come.

In the case of acoustical transducers, at least three low-power types of vibration sensing can be used for acoustic pickup. A piezo-film type transducer can be embedded directly in soft silicone. Deformation of the film by acoustic waves coupled into the silicone through the skin generate an electrical signal. Modified micro-electret microphones and modified silicon micro-microphones can also be used. Modification consists of introducing a micro-mass on the diaphragm in order to achieve sensitivity to low frequency, low amplitude vibrations.

Systems, as disclosed herein, are based on novel approaches to ultra-low power circuitry, data transmission and systems design using aggressive electrical engineering approaches. The innovations here include implementing a high functionality, ultra-small, wireless instrumentation package. Reducing power consumption during operation involves use of very low power circuits, pulsed operation for most transducers and efficient data encoding and transmission schemes.

One embodiment of a complete system includes receiver modules (one per physiological telemeter), a receiver power supply and an Ethernet interface. Miniature Ethernet interfaces are commercially available. Each receiver includes a commercial RF receiver integrated circuit and a microcontroller for decoding a data stream from the physiological telemeter and encoding it into data bytes for the Ethernet interface. To power the receivers and consolidate the individual Ethernet outputs from the receivers, a power supply and Ethernet switch can be used.

Small, surface mount implementations can be packaged in a way to survive the saline environment of a body for over two years, and some embodiments have survived much longer in test. Thus, the packaging for externally worn devices should withstand the application. Physiological telemeters can be re-useable devices. They can be maintained by simply washing them with a disinfectant after each use.

A schematic representation of the asynchronous Pulse Position Modulation concept for low power data collection is shown in FIG. 5. Such devices can float freely on the cortex of the brain and transduce neural information for prosthetic control. An asynchronous pulse position modulation telemetry technique can be used, because this form of transmission has low power requirements. Pulse position modulation is an encoding scheme in which the signal of interest is converted to a proportional time interval. Decoding such data is accomplished by generating a signal proportional to the time between pulses. Usually, two pulses are used to mark the beginning and end of the data point, which ensures a uniform sampling rate. By using a single pulse to mark the end of a previous interval and the beginning of the next interval, rather than two pulses, transmission power is reduced by 50%. While this incurs some additional overhead in reconstructing the signals, today's computers can easily handle the task. The key to achieving low power with pulse encoding is to reduce or minimize the duty cycle or on-time of the transmitter.

An adaptive pulse detector circuit can be included in the receiver section to discriminate the received pulses, e.g., short RF, acoustic, etc. The adaptive pulse detector adjusts its detection threshold, based on noise and incoming signal strength, to allow detection of the fastest rising portion of the incoming pulse. The adjustment is helpful because the pulse amplitudes are very small, and the environmental noise variations that they ride on are large, so a fixed threshold would not always be optimal or even guaranteed to work without substantial performance loss. For example, a fixed threshold would have to be set very high, but then small pulses would be detected only if they rode on sufficient noise to appear of higher amplitude. Since the adaptive pulse detector greatly enhances detection efficiency, the transmitter power output can be reduced. This ultimately reduces the required battery size or increases battery lifetime.

A low power data conditioning and multiplexing circuitry can be implemented for the physiological telemeter using commercially available low power/noise components. Under some circumstances, such circuits might not operate in the presence of an RF field without significant corruption of high impedance pathways, because RF signals might be rectified by the non-linear electrode contact impedance and/or non-linear elements within the circuits. To verify operation in the presence of an RF field, a two channel prototype telemeter was implemented using commercially available components. The transmitter and receiver modules were set to 433 MHz. A bioelectric signal amplifier and resistive strain bridge amplifier were constructed using commercially available low noise/low power op-amps. Conditioned analog signals were then converted to an RF pulse position-encoded data transmission using analog-to-digital converters and an embedded program within an RF transmitter microcontroller from MicroChip. Transmitted data pulses were received by an RF receiver from MicroChip and passed to the adaptive detection system. The signal-to-noise ratio for the hypothenar EMG was ˜25 for a bandwidth of 50-500 Hz. From the good signal-to-noise ratio, a reasonable conclusion may be made that RF interference is not an issue.

The signal conditioning, encoding, and multiplexing circuitry power consumption of the telemetry circuit is kept low by designing the circuits to operate reliably with 10-100 nA bias currents, mostly in the subthreshold region. A high speed pulse detector enables use of very short (˜100 nS) data pulses, which greatly reduces the duty cycle for the pulse output. Thus, the average current consumption can be minimized by use of a sensitive, high speed receiver. It is possible, but not necessary, that systems according to the present disclosure operate with 100 nS pulses. Pulses on the order of 1 μs in length can be used, which results in large power savings by duty cycle reduction.

Further power reductions for the integrated circuit implantable system are realized by optimizing the detection sensitivity of the receiver. For example, an adaptive threshold approach basically sets the detection threshold based on signal-to-noise of the incoming information.

Optimizing a telemeter for ultra-low power can lead to complex decoding processes. While some data decoders for demultiplexing and decoding asynchronous pulse streams can be direct digital decoders using programmable gate arrays, higher fidelity can sometimes be achieved using analog circuit techniques. These were complicated, expensive, and dedicated circuits that could be greatly simplified by using microprocessor based decoders. Recently, a microprocessor-based decoder system for single channel transmitters that outputs a decoded, demultiplexed digital representation of the original signal has been developed. This technique will be further improved to include demultiplexing of signals and applied to the decoder portion of the receiver unit in the Physiological telemeter system according to the invention.

An important concern is the size and mass of the telemeters. Telemeters should be small and have low mass to allow fixation over small regions of interest, to stay attached during high acceleration events (throwing, jumping, landing, falling, etc.) without substantially distorting and stretching the skin, which could move devices away from their target areas.

The size, mass, and lifetime of the battery used in a telemeter is determined by power consumption of the telemeter, which is dominated by the data transmission power requirements. To minimize power requirements of the telemeter, a highly sensitive, adaptive receiver is used, thereby reducing the energy in each data pulse. Data is encoded into an efficient, low duty cycle pulse format.

Because of extreme power restrictions, the maximum analog signal bandwidth transmitted by the telemeter is about 500 Hz. In one embodiment, to provide reasonable fidelity, the data is transmitted with at least about 10 bit resolution. Four sensors are included in each telemeter with an aggregate average data rate of 2 kHz. Battery voltage readout occurs as a marker bit as well, in order to maintain synchronization.

A commercially available microcontroller/RF transmitter (rfPIC from Microchip) can be used. This particular module includes a four channel analog-to-digital converter that is used to convert the analog data from the sensor conditioning circuitry to a digital word for transmission. Data is encoded for power-efficient transmission, then the data is transmitted on a unique frequency to a frequency-matched receiver. One embodiment includes one low cost receiver detector stage for every transmitter frequency of interest. The above-mentioned rfPIC transmitters/receivers can be adjusted at the circuit board level to any frequency between 315 MHz and 433 MHz by proper selection of crystal and other circuit elements. By spacing the transmitter frequencies adequately, using sufficiently long data pulses and narrow band filtering on the inputs to the RF detectors in each receiver, channel-to-channel interference can be reduced to an acceptable level.

The power consumption for the rfPIC transmitter during low power data transmission is on the order of 3 milliamps at 2.2 volts. This power usage represents transmission of ordinary analog signals, without any conversion, for example. While such a configuration is adequate for an initial implementation, battery recharge cycles would likely be limited to a few hours rather than a full day. Thus, the transmission protocol for the rfPIC transmitter/receiver is modified for asynchronous pulse-position encoding with very low duty cycles in accordance with the present invention.

An asynchronous pulse position encoding approach enables telemetry devices to consume less than 10 microamperes at 2.5 volts. Assuming an average data rate of 3 kHz (in order to achieve a minimum data rate of 2 kHz with the asynchronous approach), it is possible to use a transmission scheme with 10 μs on time and 323 μs off time to achieve a power saving, low-duty cycle. Under these assumptions, the average power consumption is 3 mA*10/333+0.5 mA*323/333=0.54 mA. Thus, telemetry devices consume less than 10 microamperes at 2.5 volts.

If all channels are sampled at a minimum frequency of 1 kHz, the aggregate minimum data rate for a 4 channel system is 4 kHz. This provides more than enough bandwidth for EEG, EKG, temperature, perfusion, and oxygenation. EMG and vibration are somewhat compromised, because there are modest amounts of signal energy above 500 Hz. Thus, it is reasonable to limit sensors that incorporate EMG or vibration sensing to two channels sampled at a minimum aggregate rate of 2 kHz. Additional bandwidth could be gained at the expense of additional power (battery size and weight) or shorter battery recharge intervals.

A modified sampling scheme to further conserve power samples the higher bandwidth channels (such as vibration and EMG) more frequently than the lower frequency channels (such as temperature, perfusion, oxygenation, EEG, EKG, etc). This can be accomplished by interleaving the lower frequency channels, such that the EMG or vibration channels are transmitted every second interval while temperature, perfusion and oxygenation are transmitted every 6^{th} interval. An example sequence is: emg-tem-emg-bat-emg-vib, where the aggregate data rate is just twice that required for EMG alone.

Typically, the batteries are charged overnight and then used during the following day. However, there may be times when the devices need to be charged in advance of use, and some form of on/off/charge switch can be incorporated, preferably still protected by the encapsulation. This switch allows power-up of a device that ran low on battery and may have entered a non-functional status.

The received data stream can be decoded and de-multiplexed by a receiver circuit tuned to a single transmitter frequency. A key to lowering transmission power consumption of the telemeters is implementation of an adaptive detection circuit, similar to that described in U.S. Pat. No. 6,898,464, hereby incorporated by reference herein. Basically the circuit determines its own threshold based on the noise floor and the incoming signal amplitude. This allows optimal detection with minimal false positives. At the data rates anticipated, inexpensive microcontrollers can perform the decoding task for a single transmitter.

Received and decoded data are directly converted into a digital format using distributed microcontrollers. Digital data is collected into a parallel bus format and transferred to an inexpensive, microprocessor based parallel-to-100 Mbps Ethernet interface. An 8-bit address (one for each potential sensor, up to 256), a 32-bit time code (about 10 uS resolution for an 8 hour day), and the 10-bit data word are sent to the Ethernet interface. For a fully-implemented system, this can result in a data rate as high as 40 Mbps, which can be readily handled by the Ethernet link. Other bus structures and data transfer options that can sustain the required data rate are also acceptable.

Inexpensive packaging of the physiological telemeters can be accomplished using existing techniques for implantable devices [26, 27] where hybrid micro-power circuit assemblies, cleaned and packaged in a custom silicone formulation, have been shown to maintain high isolation resistivity for many years while immersed directly in saline solutions. The only breaks in the encapsulation are for sensor interfaces for the bioelectrical signal pickup electrodes and the thermistor used for skin perfusion measurements. All else is embedded within the packaging. A three-part package can be used. The electronics and battery are fully sealed within the custom silicone encapsulation, which provides the primary electrical isolation from sweat, for example. An overcoat of hard polyurethane provides mechanical protection. Outside the instrumentation “nugget,” another layer of silicone is used to provide a flexible base for the biosignal electrodes, so they conform to curvatures of a body and changes in shape during activity.

The design of the electrode pick ups can be customized for EMG, EEG, EKG, EOG or whatever other application is of interest. Customization can include concentric ring (focused pickup with low cross reception), linear (traditional), or any other geometry of interest. The size and physical location of the electrodes relative to the instrument package is also easily varied. Thus, EKG and EEG electrodes can span a greater area than a small concentric ring-focused EMG pickup. The vibration sensor can be embedded within the primary instrumentation nugget, while the thermistor protrudes slightly to ensure direct skin contact.

Packaging processes may shift the center frequency of the transmitters. If this effect is determined, it can be compensated for during pre-encapsulation tuning. The batteries can be placed over or under the transmitter, or to the side without significantly affecting the received signal amplitude. In addition, proximity of the transmitter to a person tends to increase received signal strength. Thus, the loop antenna can be located around the packaged electronics, such as an out ring on the transmitter circuit board.

Analog conditioning circuitry is located on the bottom layer of a four-layer circuit board, for example, and the transmitter circuitry is located on the top layer of the circuit board. Ground and power planes are located between the top and bottom layers. The battery is located over the top of the transmitter, and the electrode pickups and other sensors are located underneath the board, with the analog conditioning circuitry.

Various conformable medical adhesive tapes (e.g., from the Johnson & Johnson or 3M companies) can be used to hold the telemeters in place during high acceleration levels.

### Use

While accurate statistics are lacking, it is generally well recognized that a large number of individuals are injured or disabled every year. Many of the injuries are caused by accidents in normal living, but many more happen during sports and recreational activities. A large percentage of injured individuals seek professional help for rehabilitation. The miniature physiological telemeter described herein can find broad application in rehabilitation settings, including exercise therapy, physical therapy, gait and motion analysis studies, etc. It can have perhaps greater impact on prevention of injury by enabling new studies in sports medicine and occupational health that currently cannot be done. Besides human subjects, all of the above uses are particularly appropriate in veterinary medicine and in monitoring animals, especially horses, in sports activities.

The physiological telemeter system can complement existing technologies, including routine physiological testing instrumentation, as well as video capture and 3-dimensional reconstruction of movement. In addition to the applications outlined here, others may be of interest to clinicians. A telemetered system can enable more routine use of EMG in diagnostics by facilitating collecting data from more normal movement paradigms. For example, Parkinsonism is often studied with the aid of EMG measurements [1], but currently must be done in controlled environments, because of the cabled systems typically used. With the disclosed system, physiology studies of muscles could be done for complex, high speed movements, without significant artifact generation by the tethered systems [2-4]. EMG analysis during natural movements can also be used as a diagnostic to determine the specific nature of an injury [5, 6].

In addition, the present invention is not limited to use with an aminate object (e.g., human, animal (mammal, bird, insect), but can be applied in monitoring a number of parameters or events in plants or inanimate objects. For example, data in locations that are impractical to reach for any reason, e.g., cost, barriers or obstructions, hazardous environments, etc., can be collected using the disclosed system and method. The low power and small size of the disclosed system permits long term monitoring of data in locations that may be moving or have no access for wires, for example, with a minimum amount of invasiveness. For example, a receiver may be placed on an opposite side of a barrier from a telemeter according to the present invention where no direct connections are available. The receiver may also be spaced from the subject to which the telemeter is coupled to avoid limiting a range of motion for the subject, as might otherwise be the case with wire connected communications. Applications can range from aircraft to industrial installations, for example. Accordingly, while the present description and following examples discus telemeters for use with a live body, which includes animals, insects and the like, the overall application of the disclosed system and method should not be limited to the same.

### Patients Undergoing Exercise Therapy

The system can find extensive application in guiding, training and monitoring patients during physical therapy and rehabilitation exercises in the clinic. Because it is inherently portable, it is useful in guiding and monitoring patients in a home or exercise facility. Also, this technology has broad significance for a variety of patient populations who have sustained disabilities and who are involved in rehabilitation programs in a clinic or, more importantly, outside a clinic. In addition to providing a source of instant feedback and guidance to patients using the system during exercising for example, the system allows logging of physiological parameters over time and specific muscle use profiles. This can be particularly important for rehabilitation of complex structures, such as a shoulder following a throwing injury, where a variety of exercises and therapies are recommended but must be performed with significant attention to detail in order to provide maximum benefit [7]. A readily available, easy to use, unobtrusive EMG telemetry system can facilitate better understanding of the exercises by the patient, as well as provide a form of biofeedback during the exercises.

A key issue with exercise therapy prescriptions is that it is not always clear that the exercises are most efficient for accomplishing the purpose. Any normal movement has a variety of muscle patterns that could be used to accomplish it. MRI studies are impractical for common use, though they provide valuable insights into which muscles are preferentially used during various exercises [8, 9]. EMG monitoring is a more practical means of identifying or monitoring many muscles during exercise [10-13]. While somewhat more difficult to achieve reliably during vigorous exercise, because of cabling issues, EMG analysis can even lead to a new understanding of physiologically distinct muscles and movement strategies. With truly wireless telemetry system, these complex questions can be investigated.

### Patients Undergoing Rehabilitation for Disabilities

The population of people with disabilities in the United States is significant. One of every seven citizens˜49 million Americans˜has some type of disabling condition. Approximately one third of these people have a disabling condition so severe that they are unable to carry out the major activities of their age group, such as working, attending school or providing self-care. About another third are restricted in their major activities, and the remaining third are limited in other types of activities. In 1992, three quarters of the all disabling conditions were due to diseases or disorders, such as emphysema, heart disease or arthritis. The economic costs of disability are enormous. Expressed in 1994 terms, the medical care expenditures (direct costs) amount to approximately $160 billion, and the indirect costs (loss of productivity) amount to approximately $155 billion—a grand total of more than 4% of the gross domestic product [14].

Rehabilitation is essential to minimize the impact of disabling conditions on quality of life, loss of productivity, and utilization of medical services. The process of rehabilitation includes the use of various forms of exercise and functional activities that alter the resting physiological state. For clinical and safety reasons, it is important to monitor a patient's physiological responses to an acute session of therapy in a laboratory, in a clinic, and in the patient's own environment. Available equipment in standard testing laboratories is too large and expensive, and it is not useful in the clinical and community setting. Monitoring patients outside the clinic and understanding their physiological responses to activity could help optimize the therapy prescription and prevent morbidity associated with dangerously high physical activity.

The disclosed technology allows unobtrusive monitoring of the heart, lungs, temperature, perfusion and oxygenation, and it can be used to detect such events as onset of overexertion, fatigue and hyperthermia, which could be quite useful in routine monitoring of patients with disabilities during rehabilitation as inpatients and outpatients. Optionally, arrhythmia detection or other enhancements can be programmed into the software package.

### Sports Medicine Studies

The multi-channel miniature physiological telemetry system is uniquely suitable for sports medicine in enabling studies of the physiology of human exertion, movement and movement disorders that cannot now be accomplished with cabled instrumentation systems. Such studies may lead to improved understanding of how the cardiopulmonary system adapts during prolonged activities, how physiologically distinct muscles are used, how they fatigue, what compensatory mechanisms take over during prolonged, physically demanding activities and effects of training, practice, etc. By monitoring multiple parameters, such as local temperature, electrical signals, perfusion and oxygenation, together with heart, lung and muscle sounds, a more complete physiological assessment can be accomplished. Where concomitant video analysis is not available, simultaneous monitoring of joint angles and mechanical event may also provide useful information, if the measurement does not interfere with the physical activity being studied [15].

Examples of potential applications are many, such as studying the physiology of high jumping, sprinting, hurdling, pole vaulting, discus, pitching, football linemen, throwing a football, basketball players, soccer players, kicking a ball, hitting a ball and numerous other activities. There is a large literature of biomechanical studies of athletes using 3D video analysis attempting to understand the relationship between how an athlete moves and probability of injury [16-22]. By combining EMG analysis with 3D video analysis, the movements and the actuators of the movements can be simultaneously studied [23, 24].

Nolan Ryan, who played major league baseball into his mid-40s, routinely threw harder than 93% of major league pitchers. It would be of great interest to understanding why Nolan Ryan and others can have long careers while involved in such physically demanding activities without chronic injury and pain. Most major league pitchers are at least temporarily disabled at some point in their careers due to injury and often perform in pain.

Better information on the timing and use of muscles during complex sports movements and development of improved training techniques may greatly reduce the large number of injured athletes. This may be particularly important for prevention of injury in youth athletes where for the most part training is based on anecdotal hearsay passed along through generation to generation of amateur coaches.

Table 2 summarizes some uses and organizations that may benefit from the present invention.

TABLE 2
Table of examples of possible uses for
the miniature physiological telemeters.

| Application | Target Organizations |
|---|---|
| General | Medical Schools |
| Rehabilitation | Occupational Therapy Centers |
| Rehabilitation | Occupational Therapy Educational |
| Rehabilitation | Outpatient Clinics Rehabilitation |
| Rehabilitation | Physical Medicine Centers |
| Rehabilitation | Physical Therapy Centers |
| Rehabilitation | Physical Therapy Educational |
| Sports Medicine | Baseball - College Teams |
| Sports Medicine | Baseball - Major/Minor/Rookie League Teams |
| Sports Medicine | Baseball - Professional Training Organizations |
| Sports Medicine | Sports Medicine Clinics |
| Surgical Recovery | Hospitals |
| Surgical Recovery | Orthopedic Centers |

### Occupational Health Studies

Repetitive stress injuries are common in sports and in a variety of occupations, from construction worker to baseball player to office worker. Such injuries are common in children, adolescents, adults and the elderly. Physical exhaustion and overheating due to excessive exertion are also common in manual laborers and athletes alike. The availability of an unobtrusive, easily applied device enables new studies that may result in improved techniques for minimizing injury and illness. It can also be useful as a routine monitoring device for athletes and workers during particularly high risk endeavors (firefighters, summer football practice, baseball spring training, etc).

The ability to simultaneously measure multiple parameters can be quite valuable in certain studies, such as studies of hand-arm vibration syndrome [25]. Simultaneous EMGs can be acquired with vibration information, if the attached devices are of low mass and capable of moving with skin to avoid vibration-induced artifacts. By attaching multiple physiological telemeters with EMG, vibration, perfusion and perhaps oxygen saturation monitoring sensors, the etiology of vibration-induced white-fingers may be better understood. The degree of propagation of the vibrations can be documented, together with responses of the musculature and local vasculature inferred from perfusion and oxygen saturation measurements.

### Multiple Related Uses

As outlined above, there are several related categories of uses for the disclosed technology. Initially, it is expected that clinical rehabilitation, sports medicine, intensive care for post surgical recovery monitoring, professional physical training (particularly professional and Olympic sports organizations and many college athlete training programs) will use the disclosed technology. However, systems will also be of interest to some high school and more college training departments, and commercial exercise centers.

The following examples are presented to illustrate the advantages of the present invention and to assist one of ordinary skill in making and using the same. These examples are not intended in any way otherwise to limit the scope of the disclosure.

### Example I

### Receiver/Decoder Units that Send Data to an Ethernet Capable Computer

Referring again to FIG. 1A, the front end of an RF receiver circuit 22 is a commercially available IC designed to operate in a 315-450 MHz band. A signal strength indicator pin (not shown) conveniently reflects the incoming power at the center frequency, so all of the RF-to-pulse translation is accomplished in this single integrated circuit (IC). The resulting waveform contains all signal pulse energy, as well as noise from other sources and is applied to an adaptive threshold detector (not shown). The adaptive threshold detector first senses the noise floor using an RMS circuit. This is equivalent to the standard deviation of the incoming signal. Then, based on an adjustable multiplier, the threshold is set. Since they are very short, the incoming pulses mixed with noise do not significantly impact the computation of the threshold. The multiplier is chosen based on the desired statistics of the detection process. This noise is generally a normally distributed amplitude variable. That is, the acceptable error rate when noise randomly crosses the threshold can be set to whatever the application requires. In practice, the normal distribution probability function is used to determine the multiplier as a starting point, then a counter measures the actual error rate for a given setting over time to establish the calibration. The detected pulses are then converted to a logic level pulses for a digital decoder 24, which consists of a 40 MHz microcontroller with a 230 kbps (kilo bits per seconds) RS232 interface. The bit rate for this data transfer is 160 kbps, assuming a 4 kHz aggregate data rate, 24-bit data/ID word, and 16-bit short term time code needed to detect data drop-outs. Software is programmed to count the time interval between pulses, detect the marker pulse (longest pulse interval), label the data with an ID and time stamp, and pass the information to an Ethernet interface. The Ethernet interface automatically manages all communications with the computer at 100 Mbps. The required Ethernet bit rate (including protocol overhead) for a 3-channel, 4-sensor physiological telemeter system is approximately 2 Mbps This requirement scales linearly with the number of channels, so a system of eight channels requires about 11 Mbps, and a system of 64 channels requires about 42 Mbps.

A full wave antenna (˜30″) with a low noise RF amplifier and band filter senses the RF signals and passes them to the receivers. The receiver includes pulse counters in the adaptive threshold detectors to determine the error rate after the system is set up. The multiplier for the adaptive threshold is adjusted until the error rate is within specifications (Table 3). This threshold setting determines the required transmitted power from the physiological telemeters, as discussed below. Test RF pulse trains with known characteristics can be used to determine the noise contribution and functionality from the receiver-to-computer data path to ensure proper function before system tests.

### Example II

### Battery Powered 4-Sensor Physiological telemeter that Operates on Three Unique RF Frequencies using Surface Mount Technology

Several approaches can be taken to reduce power requirements. One design approach is to minimize component count and use commercial rail-rail low power IC op amps, which consume less than 70 μA/OpAmp at 2 volts. Reducing the size of the circuit enhances performance, as parasitic capacitances and inductances are minimized by shorter traces.

For clinical rehabilitation, post-surgical recovery, and sports medicine applications, any device worn on the skin should be extremely small and lightweight. In order to construct an unobtrusive wearable telemeter, power consumption (hence battery and final package size and mass) should be minimized. One embodiment incorporates only essential functions into the telemeters. For example, the telemeters can function as much as possible as simple data acquisition and encoding modules with minimal signal conditioning, unless such conditioning saves power. Signal conditioning can reduce power consumption if, for example, only the average power in an input signal is needed. A good example is transmitting the average power of an EMG signal at a 50 Hz bandwidth, rather than the raw EMG signal at a 500 Hz bandwidth. This directly saves 90% of the power normally required to transmit a raw EMG signal with good fidelity.

Although signal-to-noise evaluations are somewhat subjective, the signal-to-noise ratios should be at least about 10, but could be as low as about 5 for a useful device.

The main challenge for this design is reducing the power consumption of the transmitter, because the transmitter accounts for a large fraction of the total power consumption. Before optimizing the transmitter power, several variables should to be defined. Using the adaptive pulse detector and limiting the range to 10 M allows reduction of the transmit power to a defined minimum that inherently is based on a signal-to-noise differential. Another important factor that should be defined prior to minimizing transmitter power is antenna efficiency. An antenna that is embedded in the encapsulation concentrically around the outside of the circuit is preferred from a physical/cosmetic point of view, such as is illustrated in FIGS. 3A-3C. The two antenna parameters to be optimized are transmission efficiency and directionality. In all cases, the inductances of the configurations are tuned out. Alternative antennas include a loop adjacent to the circuit and a simple short wire antenna. Transmitted RF power at a fixed distance can be compared for various antenna configurations with the transmitter taped to a saline filled phantom [28] to simulate absorptions and reflections by a body, as these will also vary with antenna configuration. Effects of orientation of the transmitter relative to the receiver can also be measured by changing the orientation of the saline phantom with the telemeter strapped in place. The most efficient antenna with the least dependence on orientation can be chosen.

Additional power savings can be realized by reducing the duration of the transmitted pulses. However, the bandwidth of a pulse is related to the length of the pulse. So, the shorter energy-conserving pulses spread energy over a greater frequency spectrum, thereby reducing the power at the center frequency, where the receiver is most sensitive. This reduces the signal-to-noise differential at the input to the adaptive detector. The signal-to-noise ratios of the pulses received can be compared to pulse durations for the chosen antenna configuration at various transmitter output powers. The duration can be set to the minimum that provides adequate signal-to-noise differential for the specified transmission error rate.

Finally, using the optimized antenna and pulse duration, the output drive to the antenna can be adjusted to allow less than 0.1 ppm error in detection at the 10 M distance, if possible within the power limits. In some embodiments, the maximum RF output power from the physiological telemeters is 15 mW, which is 1% of the current US limit and much less than cell phones.

### Example III

### Hollow Glass Spherical Filler for Packaging

Hollow glass spheres of about 100 mu. in diameter, such as those available from 3M under the trade name Scotchlite Glass Bubbles K Series and S Series, are mixed with silicone to create an encapsulation material. The hollow glass spheres are preferably packed tightly, such as by a centrifuge. Essentially, the silicon acts as an adhesive to adhere the spheres to each other. The resulting encapsulation material is quite rigid. The hollow glass spheres reduce the stretchiness, weight, density, thermal conductivity, stray capacitance and dielectric constant of the encapsulation material.

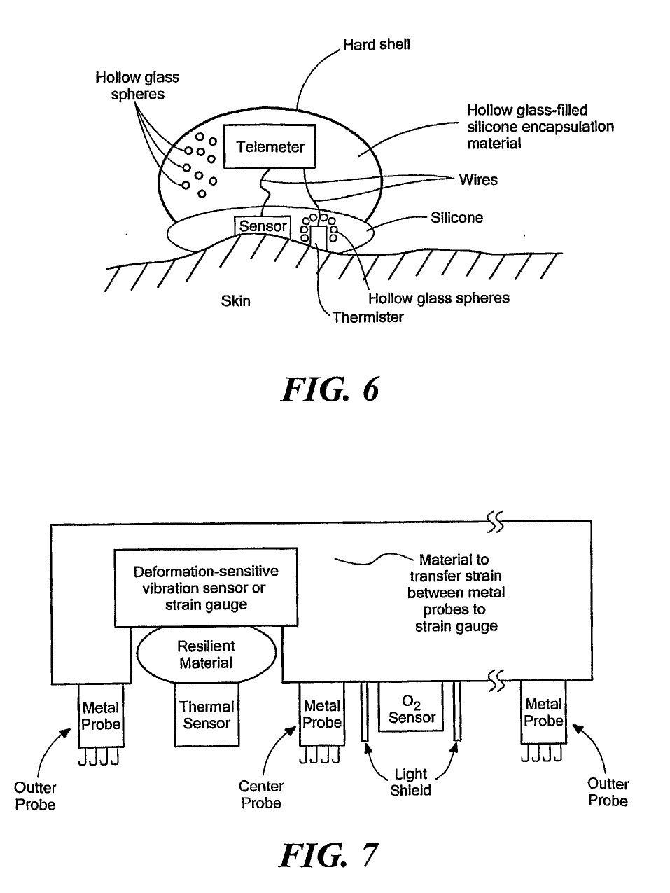

Referring to FIG. 6, the resulting encapsulation material is used to encapsulate electronic circuitry of a surface-mounted, multichannel telemeter, biometric sensors, electric wires interconnecting the biometric sensors and the telemeter and/or other components. Optionally, these elements and the encapsulation material can be further encapsulated in a hard shell. The encapsulation material (and optionally a material that subsequently forms a hard shell) can be, for example, injection molded around these elements. Such an encapsulation material holds these elements relatively rigidly, with respect to each other.

Reducing the dielectric constant facilitates assembling the telemeter complex. It is difficult or impossible to predict the effects of all the components of the telemeter complex, including the transmitters antenna, on the completed complex. Therefore, after the complex is assembled, the antenna and frequency-sensitive components of the transmitter typically need to be tuned. However, the above-described encapsulation material has a low dielectric constant, thereby minimizing the amount of tuning required as a result of the proximity of the encapsulation material to the transmitter components and the antenna.

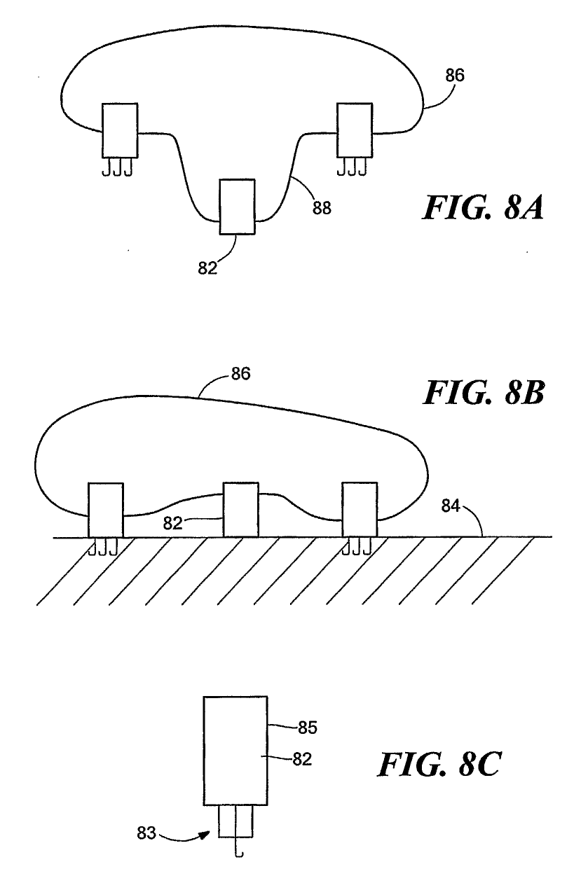

One or more telemeters are attached to the skin of a human or animal subject. Optionally, a soft silicone or urethane layer is disposed between the skin and the encapsulation material, to more easily conform to the skin and to accommodate some flexing, stretching or movement of the skin. For example, referring to FIGS. 8A-8C, a sensor or probe 82 is resiliently contacted with skin 84 due to the mechanical properties of soft silicone 86 is shaped to provide a resilient bulge 88 that permits probe 82 to retract into soft silicone 86, when pressed against skin 84. Probe 82 may be composed of any type of sensor, including a thermistor, profusion sensors such as oxygen or carbon dioxide sensors. Probe 82 may also be composed of a compressible contact 83, as illustrated in FIG. 8C. Compressible contact 83 is composed of material that is less rigid than the material used to form a body 85 of probe 82, for example. Accordingly, when probe 82 is resiliently urged against skin 84, compressible contact 83 contributes additional resiliency and flexibility to obtaining a good contact for probe 82 with skin 84. The encapsulation material is typically firmer than the silicone. Optionally, some hollow glass spheres can be included in the silicone layer adjacent the skin around a thermally sensitive sensor, such as a thermistor.

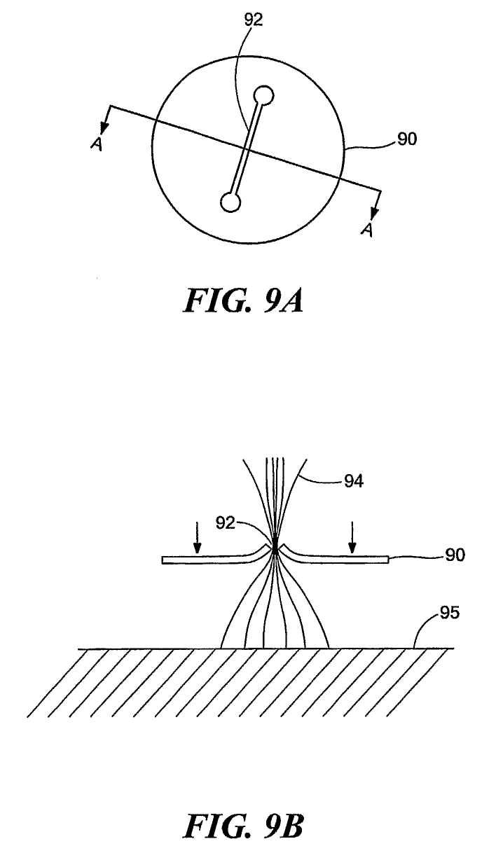

Referring now to FIGS. 9A and 9B, another type of skin attachment for a telemeter 90 is illustrated, which is especially appropriate on a hairy skin, human or animal. Surface mounted telemeter 90 includes a slit 92 that is dimensioned to accept one or more hairs 94 that act to help secure telemeter 90 to skin 95. In operation, hair 94 is pulled through slit 92 and telemeter 90 is urged into contact with skin 95. Hair 94 can be secured to prevent telemeter 90 from moving with respect to skin 95 to maintain good sensing activity. For example, an adhesive such as tape or suitable glue may be used to fix the relationship between hair 94 and telemeter 90 when telemeter 90 is placed in contact with skin 95.

Referring to FIG. 7, biometric sensors are connected to each telemeter. The sensors can include thermistors, EMG sensors, photodiodes, strain gauges, oxygen sensors, CO2 sensors and the like. The sensors are disposed near or on the skin or other portions of the body. The sensors can be disposed within the silicone or urethane layer or they can be remote from the telemeter and connected thereto by wires, optical fibers or the like. For light-sensitive sensors, such as oxygen and CO2 sensors, opaque shields can be included in the soft silicone, around the sensors. Alternatively, the soft silicon can itself be dyed to be opaque.

### Example IV

### Telemeter Attachment

Again with reference to FIG. 7, the explanted telemeters or the sensors are attached to the skin, such as by an appropriate adhesive or by hooks, which will be described here. Attachments with one or more hook-shaped portions or undercuts can be used to attach the telemeters or the sensors to the skin. For example, these attachments can be shaped like a letter “J,” like an anchor or like a 3-dimensional arrow head.

As is depicted, many such attachments can be connected to a surface of a sensor. When the sensor is pressed against the skin, the attachments penetrate the top layer(s) of the skin and hook onto tissue (such as, for example, collagen fibers), thereby mechanically attaching the sensor to the skin. Preferably, the length of the attachments is selected so the attachments enter or penetrates the epidermis, the thickness of which varies, depending on the species of the subject. For example, the attachments can be about 1 mm long. Similarly, these attachments can be connected to other components of the telemeter complex to facilitate attaching the complex to the skin.

The attachments can be made of a variety of materials, and can be made according to a variety of methods, including molding, e.g., manufacturing them in a swedging machine.

The top layer of skin sloughs off over time. Therefore, if some of the attachments break away from the sensor and remain lodged in the upper layer of the skin, these attachments will slough off with the skin.

Optionally, the attachments can provide electrical connections to the tissue, thereby obviating the need to remove nonconductive top layer(s) of the skin or to apply conductive gels between the skin and the sensor.

### Example V

### Test of a Prototype Physiological Telemeter

Several additional aspects of the design of this system were explored by designing and fabricating a concept test sensor/encoder/transmitter board. To evaluate the system, a receiver/decoder/Ethernet link was built from existing instrumentation modules. The main issues to be explored by this test board were: 1) possible RF transmitter contamination of sensitive, low power sensor conditioning circuitry; 2) possible high noise or signal distortion from the ultra-low power multiplexing/encoding process by RF fields using the optical telemetry encoding techniques; 3) possible noise induced into the receiver by the digital decoder/Ethernet link; 4) whether the microcontroller/Ethernet interface could be easily implemented; and 5) functionality of a PVDF vibration sensor embedded within a silicone electrode support material.

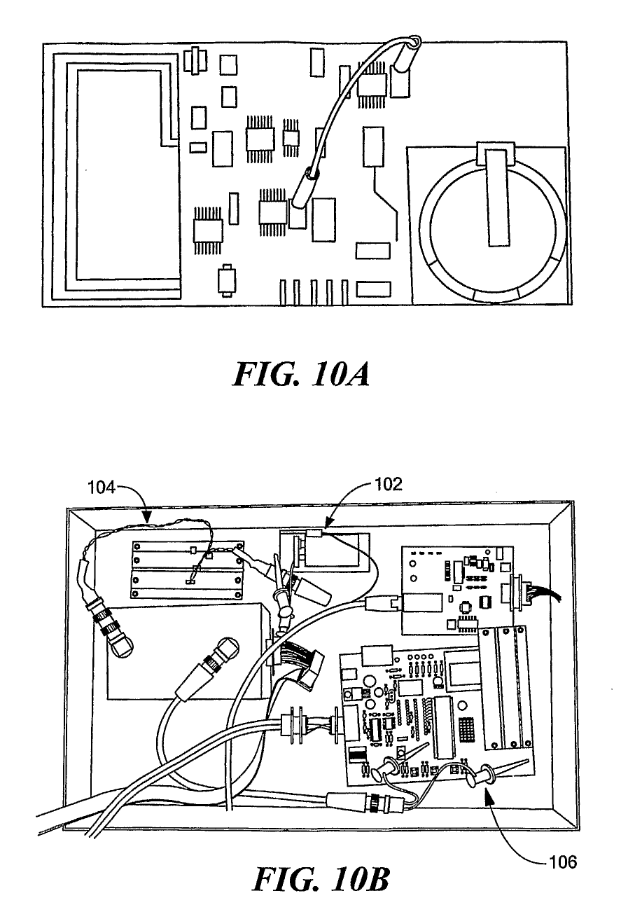

FIG. 10A is a photograph of a prototype physiological telemeter. The prototype can be used to evaluate various EMG signal conditioning circuits and potential noise issues from encoding and transmitting signals. Left to right in FIG. 10A, there is a battery, three types of experimental EMG amplifiers (selectable by jumper), temperature, vibration and power monitor conditioning circuits, a multiplexer, pulse position encoder, transmitter and loop antenna tuned to 433 MHz. In one embodiment, this unit can be condensed to approximately the diameter of a quarter, but 7 mm thick, with sensors on the skin, loop antenna around, and battery on top. This is possible by eliminating many components of this design that were included for testing, substituting SC70, TSSOP, quad packs and Chip Scale components. The layout of the components can be altered to maintain low noise conditions. A custom, micro-power integrated circuit that conditions the analog transducer signals and encodes them into a serial data stream can be fabricated using the MOSIS foundry service, for example. A commercially available chip for transmitting the data can be integrated into a miniature hybrid assembly. If a design criteria cannot be satisfied, it would be most expedient to simply substitute a larger battery and increase the transmitter power. Much of the circuitry of the devices can be implemented in a custom integrated circuit to allow reduction of the circuit area and allow conformable packaging.

FIG. 10B shows the receiver/decoder assembly used to acquire the test transmissions from the prototype telemeter example. Center top under the 9 volt battery is a 433 MHz receiver 102 with a simple wire antenna. Received signals are sent to an adaptive pulse detector 104. Time between pulses is digitized by a microcomputer 106 and sent to a commercial Ethernet interface. Data streams received by the computer via the Ethernet link are then archived or processed.

RF contamination of the signals was undetectable. The only place in the circuit where significant RF could be detected was within the low power encoder, but this did not contaminate the signal. The reasons for this are: 1) a 6-layer circuit board was used with proper attention to power and ground planes, decoupling and sensitive node shielding, 2) the battery-powered telemeter is inherently “electrically floating,” which provides extremely high common-mode rejection for most environmental noise sources, 3) the RF frequency is many orders of magnitude above the cutoff for the sensor instrumentation, and 4) the RF is on only when the encoder is in a reset state, so the encoding process does not take place in the presence of the intense RF fields.

Noise contributions from the sensor conditioning amplifiers were predicted in the commercial circuit specifications (˜4 μV_{rms-RTI}). By also implementing the Ethernet link, signals that had passed through the complete telemetry system were acquired. Signals from the power supply monitor (inherently low noise source) showed that contribution of noise by the entire encoding/acquisition process was less than 1 mVᵣₘₛ which is equivalent to less than 1 μV_{rms-RTI} for the EMG and Vibration amplifiers (divide by gain of amplifiers to get input referred noise). Total harmonic distortion (THD) was less than 0.5% for the EMG pathway, probably due to the slight aliasing effect caused by a marginal sampling rate evidenced by the rolling waveform. The encoder/multiplexer/transmitter processes implemented with commercial elements did not inherently corrupt the signals.

Any aliasing effects may be remedied by increasing the sampling rate at the expense of additional power. Temperature readout and power supply monitoring exhibited expected responses, and EMG signals were similar to those obtained previously.

Table 3 lists some exemplary design parameters, although other parameters are acceptable.

TABLE 3
Summary of exemplary design parameters.
Exemplary physiological telemeter Design Parameters

| Device | Parameter | Acceptable | |
|---|---|---|---|
| Physiological telemeter All | Thickness | 10 | mm |
| | Mass | 15 | gm |
| | Diameter | 35 | mm |
| | Battery Capacity | 75 | mA-hr |
| | Battery Diameter × Thickness | 20 × 1.6 | mm |
| | Total Harmonic Distortion for System | <1% | |
| | Equiv. RMS Noise from Encoder to | 5 | mV |
| | Supply Voltage | 2.5 | V |
| | Total Supply Current | 5 | mA |
| EMG Amplifier | Mid-Band Gain | 1000 | |
| | Bandwidth | 20-450 | Hz |
| | Input Dynamic Range | 50-500 | μVpp |
| | Equivalent Input RMS Noise | 10 | μV |
| | Supply Current | 100 | μA |
| Vibration Sensor | Mid-Band Gain | 1000 | |
| | Bandwidth | 1-100 | Hz |
| | Input Dynamic Range(from PVDF) | 50-500 | μVpp |
| | Equivalent Input RMS Noise | 10 | μV |
| | Supply Current | 100 | μA |
| Temperature Sensor | Transfer Function | 100 | mV/° C. |
| | Bandwidth | 1 | Hz |
| | Input Dynamic Range | 0-45° | C. |
| | Equivalent RMS Temperature Noise | 0.5° | C. |
| | Supply Current | 100 | μA |
| Battery Voltage Sensor | Mid-Band Gain | 1 | |
| | Bandwidth | 1 | Hz |
| | Input Dynamic Range | 1.5-3.5 | V |
| | Equivalent Input RMS Noise | 5 | mV |
| | Supply Current | 100 | μA |
| Transmitter | Regulatory | Per FCC | |
| | Pulse detection error rate | <10 | ppm |
| | Frequency Range | 315 | MHz |
| | Number of Frequencies (Phase-I) | 3 | |
| | Transmit Power for 10M Range | 0 | dBm |
| | Supply Current | 4.6 | mA |

For the receiver system, the specifications can be set by the commercial products to be used, such as the Microchip microcontroller, which can stream 230 kbaud data on each receiver's RS232 bus to a 100 Mbps Ethernet interface unit. Since an implementation of 64 channels requires a 40 Mbps Ethernet interface, there is no issues with data transfer for a 3-channel or an 8-channel system. A modern PC-type computer that is Ethernet capable is adequate. Data is organized on the computer to ensure easy importation to common data bases and analytical software. Software acquires the information and stores it in appropriate data files. Incoming data is flagged with the unique IP address from the respective receiver's Ethernet interfaces. Data from the four sensors is labeled by the microcontroller described above, which facilitates proper sorting. A post-processing routine for reconstructing the signals corrects the raw data for timing skew (each raw data point occurs at the end of the sampling period, and because of the pulse position modulation, the sampling period varies as a function of signal amplitude), and interpolates and re-samples the data into another file set in “comma separated value” (CSV) format compatible with most commercial software packages.

### REFERENCES

1. Carboncini, M. C., et al., The relation between EMG activity and kinematic parameters strongly supports a role of the action tremor in parkinsonian bradykinesia. Mov Disord, 2001. 16(1): p. 47-57.
2. Jones, K., et al., The effects of varying resistance-training loads on intermediate- and high-velocity-specific adaptations. J Strength Cond Res, 2001. 15(3): p. 349-56.
3. Ives, J. C., L. Abraham, and W. Kroll, Neuromuscular control mechanisms and strategy in arm movements of attempted supranormal speed. Res Q Exerc Sport, 1999. 70(4): p. 335-48.
4. Yamazaki, Y., et al., Rapid acceleration of the lower arm correlates with agonist EMGs during the initial phase. Hum Mov Sci, 2002. 21(2): p. 259-72.
5. McHugh, M. P., et al., Electromyographic Predictors of Residual Quadriceps Muscle Weakness after Anterior Cruciate Ligament Reconstruction. Am J Sports Med, 2002. 30(3): p. 334-339.
6. Kvist, J. and J. Gillquist, Sagittal Plane Knee Translation and Electromyographic Activity During Closed and Open Kinetic Chain Exercises in Anterior Cruciate Ligament-Deficient Patients and Control Subjects. Am J Sports Med, 2001. 29(1): p. 72-82.
7. Wilk, K. E., K. Meister, and J. R. Andrews, Current Concepts in the Rehabilitation of the Overhead Throwing Athlete. Am J Sports Med, 2002. 30(1): p. 136-151.
8. Horrigan, Magnetic resonance imaging evaluation usage associated with three exercises for rotator cuff rehabilitation. Med Sci Sports Exerc, 1999. 31(10): p. 1361-1366.
9. Takeda, Y., et al., The Most Effective Exercise for Strengthening the Supraspinatus Muscle: Evaluation by Magnetic Resonance Imaging. Am J Sports Med, 2002. 30(3): p. 374-381.
10. Decker, M. J., et al., Serratus Anterior Muscle Activity During Selected Rehabilitation Exercises. Am J Sports Med, 1999. 27(6): p. 784-791
11. Decker, M. J., et al., Subscapularis Muscle Activity during Selected Rehabilitation Exercises. Am J Sports Med, 2003. 31(1): p. 126-134.
12. Hintermeister, R. A., et al., Electromyographic Activity and Applied Load During Shoulder Rehabilitation Exercises Using Elastic Resistance. Am J Sports Med, 1998. 26(2): p. 210-220.
13. Mirzabeigi, E., et al., Isolation of the Vastus Medialis Oblique Muscle During Exercise. Am J Sports Med. 1999. 27(1): p. 50-53.
14. Enabling America: Assessing the Role of Rehabilitation Science and Engineering. 1997, Institute of Medicine Report: Washington, D.C.
15. Hore, J., et al., Control of finger grip forces in overarm throws made by skilled throwers. J Neurophysiol, 2001. 86(6): p. 2678-89.
16. Dillman, C. J., G. S. Fleisig, and J. R. Andrews, Biomechanics of pitching with emphasis upon shoulder kinematics. J Orthop Sports Phys Ther, 1993. 18(2): p. 402-8.
17. Werner, S. L., et al., Relationships between Throwing Mechanics and Shoulder Distraction in Professional Baseball Pitchers Am J Sports Med, 2001. 29(3): p. 354-358.
18. Fleisig, G. S., et al., Biomechanics of overhand throwing with implications for injuries. Sports Med, 1996. 21(6): p. 421-37.
19. Fleisig, G. S., et al., Kinetics of baseball pitching with implications about injury mechanisms. Am J Sports Med, 1995. 23(2): p. 233-9.
20. Werner, S. L., et al., Biomechanics of the elbow during baseball pitching. J Orthop Sports Phys Ther, 1993. 17(6): p. 274-8.
21. Fleisig, G. S., et al., Kinematic and kinetic comparison of baseball pitching among various levels of development. J Biomech, 1999. 32(12): p. 1371-5.
22. Escamilla, R. F., et al., Kinematic comparisons of 1996 Olympic baseball pitchers. J Sports Sci, 2001. 19(9): p. 665-76.
23. Kelly, B. T., et al., Electromyographic Analysis and Phase Definition of the Overhead Football Throw. Am J Sports Med, 2002. 30(6): p. 837-844.
24. Colby, S., et al., Electromyographic and Kinematic Analysis of Cutting Maneuvers: Implications for Anterior Cruciate Ligament Injury. Am J Sports Med, 2000. 28(2): p. 234-240.
25. Starck, J., High impulse acceleration levels in hand-held vibratory tools. An additional factor in the hazards associated with the hand-arm vibration syndrome. Scand J Work Environ Health, 1984. 10(3): p. 171-8.
26. Edell, D. J. and K. K. Gleason, Insulating Biomaterials Contract #NO1-2-2347. 2003, InnerSea Technology, Inc.
27. Webster, J. G., Medical Instrumentation. 3rd ed. 1998, New Jersey: Wiley.
28. Siwiak, K., Radiowave Propagation and Antennas for Personal Communications. 2nd ed. 1998, Norwood: Artech House.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. An apparatus for measuring a parameter of a subject and providing data communications related to the parameter, comprising:
a transducer coupled to the subject for sensing the parameter and providing a signal with characteristics related to a parameter value;
a converter coupled to the transducer for converting the signal to a pulse stream thereby forming a low power representation of the signal;
an omnidirectional transmitter coupled to the converter for transmitting the low power representation;
a receiver spaced from the subject and arranged to receive the transmitted low power representation from the transmitter.

2. The apparatus according to claim 1, wherein the transducer, converter and transmitter form a telemeter.

3. The apparatus according to claim 2, wherein the telemeter is located external to the subject.

4. The apparatus according to claim 2, wherein the telemeter is enclosed within protective package.

5. The apparatus according to claim 4, wherein the telemeter is located internal to the subject.

6. The apparatus according to claim 2, wherein the telemeter further comprises a self-contained power source.

7. The apparatus according to claim 4, wherein the telemeter further comprises a self-contained power source located within the protective package.

8. The apparatus according to claim 2, wherein the telemeter has a greatest dimension less than about 25 mm.

9. The apparatus according to claim 4, wherein the telemeter has a greatest dimension less than about 30 mm.

10. The apparatus according to claim 2, wherein the telemeter is shaped as a disk with a diameter of less than about 25 mm and a thickness of less than about 5 mm.

11. The apparatus according to claim 2, wherein the telemeter further comprises:
another transducer coupled to the subject for sensing another parameter of the subject and providing another signal with characteristics related to another parameter value; and
a multiplexer coupled to the transducers to selectively apply the respective signals to the converter.

12. The apparatus according to claim 2, further comprising:
another telemeter with another omnidirectional transmitter operating in a different communication range from the other transmitter; and
the receiver being operable to receive low power representations from both transmitters.

13. The apparatus according to claim 1, wherein the signal is a current.

14. The apparatus according to claim 13, wherein the converter further comprises a charge integration circuit to contribute to converting the current to the pulse stream.

15. The apparatus according to claim 13, wherein the pulse stream is formed as an asynchronous pulse stream that encodes a value of the current.

16. The apparatus according to claim 14, wherein the pulse stream is formed as an asynchronous pulse stream that encodes a value of the current.

17. The apparatus according to claim 6, wherein telemeter power is less than about 0.1 W.

18. The apparatus according to claim 6, wherein telemeter power is less than about 0.025 W.

19. A method for measuring a parameter of a subject and providing data communications related to the parameter, comprising:
sensing the parameter to provide a signal with characteristics related to a parameter value;
converting the signal to a pulse stream thereby forming a low power representation of the signal;
transmitting the low power representation with an omnidirectional transmitter; and
receiving the low power representation at a receiver spaced from the subject.

20. The method according to claim 19, further comprising locating the transmitter internal to the subject.

21. The method according to claim 20, further comprising enclosing the transmitter within a protective package.

22. The method according to claim 21, further comprising locating a self-contained power source for powering the transmitter within the protective package.

23. The method according to claim 19, further comprising:
sensing another parameter of the subject to provide another signal with characteristics related to a value of the another parameter; and
selectively converting the one and another signals to respective pulse streams thereby forming a plurality of low power representations.

24. The method according to claim 19, further comprising:
providing another omnidirectional transmitter operating in a different communication range from the other transmitter; and
receiving transmissions from both transmitters at the receiver.
